(19) United States
(12) Patent Application Publication
(10) Pub. No.: US 2013/0312764 A1
(43) Pub. Date: Nov. 28, 2013

(54) Composition For Use In Occluding A Body Duct In A Living Creature

(71) Applicant: Urogyn B.V., Nijmegen (NL)

(72) Inventor: Jan Albert de VRIES, Zelhem (NL)

(21) Appl. No.: 13/900,792

(22) Filed: May 23, 2013

(30) Foreign Application Priority Data
May 23, 2012 (NL) 2008860

(51) Int. Cl. A61L 31/06; B67D 7/74; A61F 6/22

(52) U.S. Cl. CPC A61L 31/06; A61F 6/22; B67D 7/74
USPC 128/831; 528/10; 523/121; 523/113; 222/145.5

(57) ABSTRACT

A composition for use in occluding a body duct, such as female sterilization or blocking the urethra, in a living creature with a plug prepared in situ from a composition of an addition curable polysiloxane system.

### CROSS-REFERENCE TO RELATED APPLICATIONS

This application claims the benefit of NL Patent Application Serial No. 2008860, filed on 23 May 2012, the benefit of the earlier filing date of which is hereby claimed under 35 USC §119(a)-(d) and (f). This application is hereby incorporated in its entirety as if fully set forth herein.

### BACKGROUND OF THE INVENTION

1. Field of the Invention

The present invention relates generally to a composition for use in occluding a body duct in a living creature, in particular sterilization of a female by blocking of the Fallopian tubes and in treating patients having a (temporarily) suprapubic catheter by blocking the urethra.

2. Description of Related Art

U.S. Pat. No. 4,245,623 discloses a non-surgical sterilization method of blocking or plugging Fallopian tubes of a female using a hysteroscope. A plug is formed in situ in a Fallopian tube from a medically inert polymer (elastomer) having about the same modulus of elasticity as the Fallopian tube, when cured. This block forms a safe barrier between an egg-cell and a sperm cell, thereby effectively sterilizing the female thus treated. A useful starting material is said to be a medical grade silicone elastomer, e.g. a hydroxyl-end blocked poly (dimethyl siloxane) which contains propyl orthosilicate as a cross-linking agent, and a silica filler (diatomaceous earth). The composition also comprises tin (II) octoate. During reaction (a condensation polymerization) propanol is split off. A diluent like end-blocked poly (dimethyl siloxane) having a 0.2 poise viscosity is present to reduce the viscosity of the composition. The composition also comprises a radiopaque material to permit x-ray visualization of the position of the block(s) in the Fallopian tubes.

Although this kind of sterilization has proven to be reliable, some drawbacks have been discovered. One drawback concerns the dosing ratio of polymer:catalyst in the composition, which is in the order of 1000:1. As the starting composition is to be prepared shortly in advance of the actual application such as injection, otherwise (partial) curing would have rendered the material unworkable, in an appropriate amount typically in the ml range, the dosing amount of the catalyst is in the microliter range. Any unintended deviation affects the actual curing time and other properties, which is rather unpredictable and therefore undesired. Thus precise control of the crosslinking reaction during application is difficult to achieve. Another drawback relates to the fact that some kind of shrink occurs during crosslinking. This shrink could cause voids to be present in the plug as obtained, thereby affecting its contraceptive function. Furthermore it has appeared that the crosslinked material is susceptible to degradation over time in the presence of e.g. oxygen, resulting in gradually increasing brittleness. If the material becomes too brittle, fractures could be initiated and small pieces may be separated, thereby also reducing the blocking function of the plug. This known material also requires storage at low temperatures (−18° C.) in a freezer in a protective atmosphere.

Therefore, it is an object of the present invention to provide a composition for use in occluding a body duct, which allows accurate control of its curing time.

Another object of the present invention is to provide a composition for said use, which is stable in time after curing.

Yet another object of the present invention is to provide a composition for said use, which is easy to compose from the individual components thereof.

A further object of the present invention is to wherein one or more of the drawbacks mentioned above are reduced or eliminated.

Still another object of the present invention is to provide a method of occluding a body duct in a living creature with a stable plug prepared in situ and having a long-lasting effect

### BRIEF SUMMARY OF THE INVENTION

Briefly described, in a preferred form, the present invention comprises a composition for use in occluding a body duct in a living creature with a plug prepared in situ from a composition comprising an addition curable polysiloxane system.

In the context of this application the expression “body duct” is used to define a tract or channel in the body, that supplies or discharges body cells and/or body fluids and the like, contrary to a random cavity in the body that has no transporting function.

The present inventor has discovered that many of the drawbacks described above relate to the curing mechanism of the polysiloxanes. The composition disclosed in U.S. Pat. No. 4,245,623 is crosslinked through a so called condensation cure mechanism, wherein upon crosslinking propanol is split out. This is believed to be a main cause of the shrink of the known material. Contrary to a condensation cure mechanism, a composition according to the invention is crosslinked by means of an addition cure mechanism, wherein the polymer base, a polysiloxane, reacts with a crosslinker without generating a byproduct. Such a byproduct is considered to be responsible for shrink and degradation over time. In addition, addition curable polysiloxane compositions according to the invention can be prepared in a 1:1 ratio from the components that are contained in a two container mixing and dispensing system having containers of equal volume. In such a system the almost equal volumes of the starting components can be maintained separately until needed.

The starting materials of this system do not need to be stored at low temperatures. Usually the addition curable polysiloxane system can be processed up to about 10-20 minutes, e.g. 15 minutes. Thereafter curing has advanced to such an extent that the viscosity becomes too high for injection. Injection is usually monitored, e.g. using echoscopy or by X-ray imaging. Once cured, the addition cured polysiloxane composition provides a plug. The composition is less susceptible to breakdown over time, as a result stability and effect are long-lasting.

The invention also provides a prepackaged addition curable polysiloxane system for use in occluding a body duct in a living creature with a plug prepared in situ from a composition comprising an addition curable polysiloxane system, comprising a two component dispensing device having two containers separated from each other by a temporary seal. In a first container the catalyst is present, the other one comprises the crosslinker of the addition curable polysiloxane reactive system. The base polysiloxane(s) may be present in both containers.

Another aspect of the invention is the use of an addition curable polysiloxane system in manufacturing a composition for use in a occlusion method of a body duct in a living creature, in particular a sterilization method of a female by blocking the Fallopian tubes thereof, or blocking the urethra in case of a suprapubic catheter, with a plug prepared in situ from a composition comprising said addition curable polysiloxane system.

The invention also comprises a method of occluding a body duct in a living creature, in particular a sterilization method of a female by blocking the Fallopian tubes thereof, or blocking the urethra in case of a suprapubic catheter, comprising a step of applying an addition curable polysiloxane composition comprising an ethylenically unsaturated moiety terminated polysiloxane, a crosslinker and a catalyst in effective amounts for allowing an addition curing in situ, in the respective body duct and a step of allowing said composition to cure to a plug.

In an exemplary embodiment, the present invention is an occlusive composition comprising an addition curable polysiloxane system. The addition curable polysiloxane system can comprise a polysiloxane, a catalyst, and a crosslinker. The addition curable polysiloxane system can further comprise one or more of a tracer material, a bactericidal agent and a filler.

The addition curable polysiloxane system can comprise a polysiloxane having an ethylenically unsaturated moiety, an ethylenically unsaturated moiety terminated polysiloxane, a vinyl terminated polysiloxane, a vinyldialkyl terminated dimethyl polysiloxane, and/or a vinyldimethyl terminated polysiloxane.

The addition curable polysiloxane system can comprise a Pt catalyst, a Pt(0) organic complex, and/or a Pt(0) vinyl siloxane complex.

The addition curable polysiloxane system can comprise a hydrosilicone crosslinker, hydrogen dialkyl terminated siloxy groups, and/or hydrogen dimethyl terminated siloxy groups.

The addition curable polysiloxane system can comprises a first container with a catalyst, and a second container with a crosslinker, wherein at least one of the first and the second container further comprise a polysiloxane.

In another exemplary embodiment, the present invention can comprise an occlusive composition comprising an addition curable polysiloxane system, wherein the addition curable polysiloxane system comprises a polysiloxane, a catalyst, a crosslinker, and optionally, one or more of a tracer material, a bactericidal agent, and a filler.

The polysiloxane can comprise an ethylenically unsaturated moiety terminated polysiloxane, the catalyst can comprise a Pt(0) vinyl siloxane complex, and the crosslinker can comprise hydrogen dimethyl terminated siloxy groups.

The tracer material can comprise titanium dioxide.

The bactericidal agent can comprise Ag⁺ zeolite.

In another exemplary embodiment, the present invention comprises a first container comprising a portion of the polysiloxane comprising a polysiloxane having an ethylenically unsaturated moiety and the catalyst, and a second container comprising a portion of the polysiloxane comprising a polysiloxane having an ethylenically unsaturated moiety, and the crosslinker. The containers can be parts of a two component dispensing device having the two containers separated from each other by a temporary seal. The two component dispensing device can further comprise a mixing means. The mixing means can comprise a static mixer and/or catheter.

In another exemplary embodiment, the present invention can comprise an occluding method for occluding a body duct in a living creature comprising preparing a plug in situ from a composition of addition curable polysiloxane system and blocking the body duct with the plug. Blocking the body duct with the plug can comprise blocking a Fallopian tube in a female with the plug. Blocking the body duct with the plug can comprise blocking the urethra with the plug.

In another exemplary embodiment, preparing the plug comprises applying the composition of addition curable polysiloxane system, applying a catalyst, applying a crosslinker, and curing the plug, in situ, in the body duct.

The method can further comprise monitoring one or more of the preparing and blocking steps. The monitoring can comprises monitoring via X-ray imaging and/or echoscopy.

Further objects of the present invention arise from the following statements and the attached claims.

### DETAILED DESCRIPTION OF THE INVENTION

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

The composition according to the invention is applied to the body duct and cures in situ by means of a so called addition cure mechanism to a plug. Basically this mechanism involves the addition reaction of polyfunctional silicon hydride to unsaturated groups in polysiloxane chains. Although the E-modulus of the cured material is higher than the E-modulus of the known polysiloxane based materials, the cured material of the plug maintains a certain degree of flexibility due to the relatively long chains. The plug is non-resorbable.

In a preferred embodiment the addition curable polysiloxane system comprises a polysiloxane having ethylenically unsaturated moieties. The ethylenically unsaturated moiety or moieties can be present on an end Si, but also as a substituent to the polysiloxane. Vinyl and unsaturated aromatics such as phenyl are preferred examples of these moieties. Preferably an ethylenically unsaturated moiety terminated polysiloxane, more preferably a vinyl terminated polysiloxane such as vinyldialkyl terminated dimethyl polysiloxane, even more preferably a vinyldimethyl terminated dimethyl polysiloxane is used in the addition curable polysiloxane system. The above polysiloxanes are commercially available from several manufacturers including Nusil, Dow Corning and UCT.

The addition curable polysiloxane reactive system also comprises a catalyst, in particular a precious metal catalyst, preferably Pt. More preferably this catalyst is present as an organic Pt(0) complex such as a Pt(0) vinyl siloxane complex.

The addition curable polysiloxane reactive system also comprises a crosslinker for addition curing of the base polymeric polysiloxanes described above. Typically the crosslinker will be a hydrosilicone crosslinker having a number of hydrogen atoms capable of reaction with the ethylenically unsaturated groups of the base polymer(s). Preferably, the crosslinker comprises hydrogendialkyl terminated siloxy groups, preferably hydrogendimethyl terminated siloxy groups. Another preferred type of crosslinker is polyalkyl hydrosiloxanes wherein hydrogen is present in each unit of the siloxane chain. Polymethylhydrosiloxanes are an example. The crosslinker will hydrosilylate olefins in the presence of precious metal catalysts, such as Pt.

A system according to the invention is typically such that the viscosity thereof allows extrusion through a catheter that is inserted into the body duct, and such that easy application is possible. After in vivo curing the plug obtained effectively seals the respective duct. Curing takes place at low temperature. During in vivo curing the local temperature will not rise higher than 40° C., in particular not higher than body temperature, so that no damage occurs.

Typically the addition curable polysiloxane system also comprises a tracer, such as a X-ray radiopaque material or a material that can be detected by ultrasound e.g. echoscopy, in order to monitor the progress of the treatment of the invention. Suitable tracer materials include silver powder, barium sulphate, bismuth trioxide, zirconium dioxide, tantalum or titanium powders or fibers, calcium sulphate, calcium phosphate, hydroxyapatite, tri calcium phosphate, and other medically appropriate opacifier agents. A preferred tracer material is titanium dioxide, because it is easily monitored using ultrasound, while the X-ray detection properties are also fair. Moreover it does not settle in the base composition parts contrary to silver powder and barium sulphate. Echoscopy has proven to enable monitoring the treatment progress. Compared to X-ray monitoring echoscopy is less demanding to a patient. In a further preferred embodiment a bactericidal agent is also present in the composition. Silver powder is an example thereof. Preferably the bactericidal agent comprises an Ag⁺ zeolite, especially an Ag⁺ nano zeolite. Such a zeolite does not settle, allows ion exchange, e.g. substitution of silver ions by sodium ions from the body. This is a relatively slow process thereby effectively extending the time period during which Ag ions are released. Furthermore this bactericidal agent prevents the formation of a so called biofilm affecting adhesion and promoting inflammation risk.

A filler like silicate could also be present in the addition curable polysiloxane system according to the invention. Like the particulate tracer and bactericidal agent the non-resorbable filler particles are encapsulated in the cured polymeric matrix such that they cannot migrate through the body.

The composition according to the invention can be used for occluding body cavities, where passage of body cells like egg-cells or sperm, or flow of body fluids like urine is to be blocked. The plug obtained by addition cure polymerization from the composition according to the invention effectively seals the body duct. A preferred embodiment of such occlusion is sterilization of women, where the composition is inserted into the Fallopian tubes in order to prepare a fluid tight plug therein after curing. Another preferred embodiment is treatment of a patient having a suprapubic catheter. Such patients having a catheter connected to the bladder through the abdominal wall suffer sometimes from uncontrolled urine leakage via the urethra, which is highly undesirable. A plug made in the urethra from a composition according to the invention prevents this leakage. Other examples include blocking of blood veins, varicose veins, arteries supplying a tumor causing its dead. Blocking of a body duct can be temporarily or permanently.

The composition is advantageously packaged as a kit of parts, comprising at least a first container with a catalyst, and a second container with the cross-linking agent, wherein advantageously the base polysiloxane polymer(s) is/are present in both containers. An example is a double barreled syringe having parallel containers. After filling the package and contents are subjected to a sterilizing treatment. It has appeared that sterilizing using ethyleneoxide as a sterilizing agent is effective.

In a further preferred embodiment the addition curable polysiloxane system is provided as a two component system, comprising as part A in a first container:
a polysiloxane having an ethylenically unsaturated moiety,
a catalyst,
optionally a radiopaque material, a bactericidal agent and/or a filler and as part B in a second container;
a polysiloxane having an ethylenically unsaturated moiety,
a crosslinker,
optionally a tracer material, a bactericidal agent and/or a filler.

A preferred embodiment of a two component dispensing device comprises a double barreled syringe having two parallel cylindrical compartments, each provided with a plunger, the ends thereof outside the compartments are coupled. The outlets of each compartment exit into a mutual outlet, which before actual use is preferably provided with a static mixer. A small diameter tube such as a catheter, or a needle assembly is attached to the static mixer.

Instead of a static mixer a two component injection device including an internal mixer, as for example disclosed in PCT/NL2004/000827 and PCT/NL2005/000268, can be used. The preferred, advantageous and/or beneficial embodiments of the various components of the composition similarly apply to this embodiment. In order to simultaneously eject the contents of the compartments the syringe can be placed into an auxiliary device, which is able to actuate the plungers simultaneously. Usually this auxiliary device is able to move the plungers over a predetermined distance each time it is actuated, e.g. by a trigger. In this way each time a fixed volume from each compartment is ejected, mixed and applied to the duct in question.

In particular, the composition is packaged in a syringe having two containers temporarily sealed from each other, wherein a first part comprising the catalyst of the reactive system is present in a first container and a second part comprising the crosslinker of the reactive system is present in a second container. Preferably the first and second containers comprise, part A and part B as described above.

Advantageously the addition curable polysiloxane is applied in an effective amount for achieving a durometer of about 25-50 Shore A, a tensile strength of 400-800 psi, and elongation of 120-175%.

The invention also relates to a prepackaged addition curable polysiloxane system for use in occluding a body duct in a living creature with a plug prepared in situ from a composition comprising an addition curable polysiloxane system, comprising a two component dispensing device having two containers separated from each other by a temporary seal, wherein a first container comprises as part A:
a polysiloxane having an ethylenically unsaturated moiety,
a catalyst,
optionally a tracer material, a bactericidal agent and/or a filler and as part B in a second container;
a polysiloxane having an ethylenically unsaturated moiety,
a crosslinker,
optionally a tracer material, a bactericidal agent and/or a filler.

The preferred, advantageous and/or beneficial embodiments of the various components of the composition similarly apply to this package. Advantageously this package also comprises a mixing means, preferably a static mixer, and/or a catheter.

In order to simultaneously eject the contents of the compartments the syringe can be placed into an auxiliary device, which is able to actuate the plungers simultaneously. Usually this auxiliary device is able to move the plungers over a predetermined distance each time it is actuated, e.g. by a trigger. In this way each time a fixed volume from each compartment is ejected, mixed and applied to the duct in question.

The preferred, advantageous and/or beneficial embodiments of the various components of the composition similarly apply to this embodiment. In particular, the composition is packaged in a double barreled syringe having two containers temporarily sealed from each other, wherein part A is present in a first container and part B is present in a second container.

A further aspect of the invention concerns the use of an addition curable polysiloxane system in the manufacture of a composition for use in a occlusion method of a body duct in a living creature, in particular a sterilization method of a female by blocking the Fallopian tubes thereof, or blocking the urethra in case of a suprapubic catheter, with a plug prepared in situ from a composition comprising the addition curable polysiloxane system. The preferred, advantageous and/or beneficial embodiments of the various components of the composition similarly apply to this use according to the invention.

The invention also provides a method of occluding a body duct in a living creature, in particular a sterilization method of a female by blocking the Fallopian tubes thereof, or blocking the urethra in case of a suprapubic catheter, comprising a step of applying an addition curable polysiloxane composition comprising an ethylenically unsaturated moiety terminated polysiloxane, a crosslinker and a catalyst in effective amounts for allowing an addition curing in situ, in the respective body duct and a step of allowing said composition to cure to a plug. The preferred, advantageous and/or beneficial embodiments of the various components of the composition similarly apply to this method according to the invention. For blocking the Fallopian tubes the composition is frequently applied in an amount of 1-4 times 0.05-0.3 ml, advantageously 2-3 times 1 ml, while monitoring the application site preferably by echoscopy.

Numerous characteristics and advantages have been set forth in the foregoing description, together with details of structure and function. While the invention has been disclosed in several forms, it will be apparent to those skilled in the art that many modifications, additions, and deletions, especially in matters of shape, size, and arrangement of parts, can be made therein without departing from the spirit and scope of the invention and its equivalents as set forth in the following claims. Therefore, other modifications or embodiments as may be suggested by the teachings herein are particularly reserved as they fall within the breadth and scope of the claims here appended.

What is claimed is:

1. An occlusive composition comprising an addition curable polysiloxane system.

2. The occlusive composition of claim 1, wherein the addition curable polysiloxane system comprises:
a polysiloxane;
a catalyst; and
a crosslinker.

3. The occlusive composition of claim 2, wherein the addition curable polysiloxane system further comprises one or more of a tracer material, a bactericidal agent and a filler.

4. The occlusive composition of claim 1, wherein the addition curable polysiloxane system comprises a polysiloxane having an ethylenically unsaturated moiety.

5. The occlusive composition of claim 1, wherein the addition curable polysiloxane system comprises an ethylenically unsaturated moiety terminated polysiloxane.

6. The occlusive composition of claim 1, wherein the addition curable polysiloxane system comprises vinyl terminated polysiloxane.

7. The occlusive composition of claim 6, wherein the vinyl terminated polysiloxane comprises vinyldialkyl terminated dimethyl polysiloxane.

8. The occlusive composition of claim 6, wherein the vinyl terminated polysiloxane comprises vinyldimethyl terminated polysiloxane.

9. The occlusive composition of claim 1, wherein the addition curable polysiloxane system comprises a Pt catalyst.

10. The occlusive composition of claim 1, wherein the addition curable polysiloxane system comprises a Pt(0) organic complex.

11. The occlusive composition of claim 1, wherein the addition curable polysiloxane system comprises a Pt(0) vinyl siloxane complex.

12. The occlusive composition of claim 1, wherein the addition curable polysiloxane system comprises a hydrosilicone crosslinker.

13. The occlusive composition of claim 12, wherein the hydrosilicone crosslinker comprises hydrogen dialkyl terminated siloxy groups.

14. The occlusive composition of claim 12, wherein the hydrosilicone crosslinker comprises hydrogen dimethyl terminated siloxy groups.

15. The occlusive composition of claim 1, wherein the addition curable polysiloxane system comprises a first container with a catalyst, and a second container with a crosslinker, wherein at least one of the first and the second container further comprise a polysiloxane.

16. An occlusive composition comprising an addition curable polysiloxane system, wherein the addition curable polysiloxane system comprises:
a polysiloxane;
a catalyst;
a crosslinker; and
optionally, one or more of:
a tracer material;
a bactericidal agent; and
a filler.

17. The occlusive composition of claim 16, wherein:
the polysiloxane comprises an ethylenically unsaturated moiety terminated polysiloxane;
the catalyst comprises a Pt(0) vinyl siloxane complex; and
the crosslinker comprises hydrogen dimethyl terminated siloxy groups.

18. The occlusive composition of claim 16 comprising a tracer material comprising titanium dioxide.

19. The occlusive composition of claim 16 comprising a bactericidal agent comprising Ag⁺ zeolite.

20. The occlusive composition of claim 16, wherein the addition curable polysiloxane system comprises a first container comprising:
a portion of the polysiloxane comprising a polysiloxane having an ethylenically unsaturated moiety; and
the catalyst; and
a second container comprising:
a portion of the polysiloxane comprising a polysiloxane having an ethylenically unsaturated moiety; and
the crosslinker.

21. The occlusive composition of claim 20, wherein the containers are parts of a two component dispensing device having the two containers separated from each other by a temporary seal.

22. The occlusive composition of claim 21, wherein the two component dispensing device further comprises a mixing means.

23. The occlusive composition of claim 22, wherein the mixing means comprises a static mixer.

24. The occlusive composition of claim 22, wherein the mixing means comprises a catheter.

25. An occluding method for occluding a body duct in a living creature comprising:
preparing a plug in situ from the composition of claim 1; and
blocking the body duct with the plug.

26. The method according to claim 25, wherein blocking the body duct with the plug comprises blocking a Fallopian tube in a female with the plug.

27. The method according to claim 25, wherein blocking the body duct with the plug comprises blocking the urethra with the plug.

28. The method according to claim 25, wherein preparing the plug comprises:
applying the composition of claim 5;
applying a catalyst;
applying a crosslinker; and
curing the plug, in situ, in the body duct.

29. The method according to claim 25 further comprising monitoring one or more of the preparing and blocking steps.

30. The method according to claim 29, wherein monitoring one or more of the preparing and blocking steps comprises monitoring via X-ray imaging.

31. The method according to claim 29, wherein monitoring one or more of the preparing and blocking steps comprises monitoring via echoscopy.
